(19) United States
(12) Patent Application Publication
(10) Pub. No.: US 2005/0233990 A1
(43) Pub. Date: Oct. 20, 2005

(54) Anti-cancer agents comprising disintegrin genes and the treating methods

(76) Inventors: Yong-Serk Park, Wonju-shi (KR); Soo-In Kim, Goyang-shi (KR); Sung-Yu Hong, Seoul (KR); Yong-Doug Sohn, Yongin-shi (KR); Yang-Soo Jang, Sungnam-shi (KR); Chin-Kyu Hun, Seoul (KR); Dong-Sik Kim, Seoul (KR)

Correspondence Address: MADSON & METCALF;GATEWAY TOWER WEST, SUITE 900, 15 WEST SOUTH TEMPLE, SALT LAKE CITY, UT, 84101, US

(21) Appl. No.: 10/487,790

(22) PCT Filed: Aug. 29, 2001
(86) PCT No.: PCT/KR01/01461

§ 371 Date: Feb. 26, 2004

(51) Int. Cl. A61K 48/00; A61K 9/127

(52) U.S. Cl. 514/44; 424/450

(57) ABSTRACT

The present invention relates to liposome complex comprising a novel disintegrin, saxatilin, gene derived from Agkistrodon saxatilis and methods for curing and preventing tumors by transferring the complexes to a living body.

### BACKGROUND OF THE INVENTION

1. Field of the Invention

The present invention relates to anti-cancer agent showing the activities of inhibiting metastasis of cancer, angiogenesis and growth of cancer tissue, and a method for treating cancers by using the same. More specifically, the present invention relates to liposome complexes for inhibiting growth of cancer cells, which comprises gene of saxatilin, a novel disintegrin derived from Agkistrodon saxatilis originated from Korea, and a method for preventing or treating cancers by introducing the complexes into a living body.

2. Description of the Prior Art

A cancer is a disease that causes by very complex and various factors, being one of the main factors of death of the modems. Normal cells are transformed to cancer cells by various factors, and unlimited growth of cancer cells (differently from normal cells) is progressed to form a cancer tissue. A certain size of cancer tissue induces angiogenesis to supply necessary nutrients to the tissue, and the tissue is subjected to metastasis through the vessels formed by angiogenesis, and attached to certain organ of the body and grows. In particular, it is known that a cancer tissue by cancer cells occurred from the first place secretes a kind of inhibiting factor that can inhibit generation of cancer in another place. The initial stage of cancer, of which the size is not less than 3 mm, can be easily observed by clinical diagnosis, and can be removed by surgical operation at this stage. Even though the cancer can be surgically removed by operation, the operation has a danger of rather promoting metastasis of the cancer in case that if surgically unremoved cancer cells are in the course of metastasis. Thus, most of the patients for cancer operation should be treated by anti-cancer agent or radiotherapy, except some very specific cases, in order to prevent the secondary cancer by metastasis that may followed by the operation.

Though very effective anticancer agents have been developed and clinically used up to the present, most of them are inhibitors of cell growth which cannot distinguish cancer cells from normal cells. No anticancer agent that intercepts metastasis of cancer and inhibits growth of cancer has been developed or employed yet.

Angiogenesis means a series of processes to form new blood vessels from existing vessels, which is a normal and essential process for supply of nutrients and oxygen. Angiogenesis can be observed in recovery of injuries, woman's menstruation, proliferative retinopathy, rheumatic arthritis, ischemic cardiovascular disease, cancer, or the like. Since angiogenesis appears very characteristically during the course of growth of cancer cells and metastasis, it is known as principal purpose of treating cancer tissues capillary vessels comprised of collagen, glycoprotein and heterogeneous extracellular matrix (ECM) are formed by the effect of promoting factors for blood vessel formation such as bFGF secreted from cancer cells, and endothelial cells migrate to the capillary vessels cells by the effect of integrin to combine with the capillary vessels. In the process of angiogenesis, the cell fusion material, integrin known as αvβ₃ and αvβ5 is very important. It is reported that if treated with the disintegrin protein having amino acid sequence of Arg-Gly-Asp (RGD) which selectively antagonize integrin, angiogenesis can be inhibited.

Saxatilin is a disintegrin discovered from snakes venom originated from Korea, and has the effect of inhibiting angiogenesis. It was reported that recombinant saxatilin that has been recently expressed from E. coli inhibits angiogenesis of cancer cells mediated by αvβ3 integrin, but does not affect on the process of physiological formation of blood vessels that is indispensable for normal embryogenesis in chorioallantoic membrane (CAM). In addition, it was demonstrated that day after day inoculation of saxatilin protein inhibited growth of cancer cells and generation of blood vessels. However, this method is disadvantageous in that it is very difficult to retain constant concentration in order to keep anticancer effect with trouble of everyday injection. In order to overcome the disadvantages, simultaneous use of various anticancer treatments has been employed. However, the present inventors newly confirmed the fact that anticancer effect can be simply obtained for a long time by employing recently spotlighted genetic therapy, to develop the present invention.

### SUMMARY OF THE INVENTION

The present inventors performed intensive studies to develop a method to maintain the anticancer effect of saxatilin for a long time, and as a result, we found that if lipoplex prepared by mixing cationic liposome and cDNA of saxatilin gene is treated to cancer tissue to perform genetic therapy, growth of cancer cells can be inhibited for a long period.

The primary object of the present invention is to provide a substance for inhibiting growth of cancer cells.

Another object of the present invention is to provide a method for inhibiting growth of cancer cells by using said substance.

In order to achieve said objects, the present invention provides liposome complex (lipoplex) for inhibiting growth of cancer cells, which comprises cationic liposome and expression vector comprising saxatilin gene of sequence information 1.

The cationic liposome of the present invention is preferably one selected from the group consisting of liposome containing DMDK (lysine-aspartate-tetradecanol) and cholesterol, liposome containing DMEK (lysine-glutarate-tetradecanol) and cholesterol, liposome containing DOTMA (N-[1-(2,3-dioleyloxy)propyl]-N,N,N-triethylammonium chloride) and cholesterol, liposome containing DC-Chol (3β-[N-(N′N′dimethylaminoethane)carbamoyl]cholesterol) and DOPE (dioleoylphosphatidylethanolamine), and liposome containing DOTAP (1,2-dioleoyloxypropyl-3-N,N,N-trimethylammonium chloride) and cholesterol. The saxatilin gene of the present invention comprises not only the base sequence of sequence information 1, but also its mutants and its active segments.

As the expression vector of the present invention, any conventional expression vectors can be used. Particularly, an eukaryotic expression vector is preferably one selected from the group consisting of pAAV-CMV and pFLAG-CMV-1.

In addition, the present invention provides a method for inhibiting growth of cancer cells by using saxatilin gene, which comprises steps of mixing cholesterol with DOTAP, suspending the mixture in aqueous medium to prepare cationic liposome and preparing expression vector for expressing saxatilin by introducing saxatilin gene of sequence information 1 to eukaryotic expression vector; mixing the cationic liposome and said expression vector in aqueous medium and homogenizing to prepare lipoplex; and introducing the lipoplex to a cancer tissue.

### BRIEF DESCRIPTION OF THE DRAWINGS

The present invention will be explained in terms of exemplary embodiments described in detail with reference to the accompanying drawings, which are given only by way of illustration and thus are not limitative of the present invention:

FIG. 1 shows gene structures of expression vector pAAV-CMV (a) and pFLAG-CMV-1 (b).

FIG. 2 shows preparation of saxatilin expression vectors (pAAV-CMV-saxatilin and pFLAG-CMV-1 saxatilin) by inserting saxatilin gene in the expression vector of FIG. 1.

FIG. 3 is a photograph to show in vitro expression of saxatilin in 293 cells. The plasmid was introduced to cells via cationic liposome based on DOTAP. Lane 1 shows the marker of molecular weight, Lane 2 shows non-intorduced medium as negative control, and Lane 3 shows pFLAG-CMV1-saxatilin.

FIG. 4 is a photograph to show the effect of saxatilin gene on inhibiting growth of cancer cells. In the figure, A represents control group treated with B16BL6, cells of lung cancer of mouce, and PBS, while B represents experimental group treated with pAAV-CMV-saxatilin (25 μg/mouse).

FIG. 5 is a photograph to show the effect of saxatilin gene on inhibiting metastasis of cancer. In the figure, A represents control group to which PBS was injected, while B represents experimental group to which pAAV-CMV-saxatilin was treated, after injecting B16BL6 cells to mice.

FIG. 6 shows inhibition of growth of BCE cells by saxatilin expressed in 298 cells. In the figure, BCE cells are treated with saxatillin expressed in 293 cells which were transfected with pAAV-CMV1-saxatilin for 72 hours in the presence of 1 ng/ml of bFGF, and inhibition of proliferation depending on saxatilin concentration was analyzed.

### DETAILED DESCRIPTION OF THE PREFERRED EMBODIMENTS

Now, the process for inhibiting growth of cancer cells by using saxatilin gene according to the present invention is described in more detail step by step.

Step 1: Preparation of Cationic Liposome

Cholesterol is mixed with DOTAP (1,2-dioleoyloxypropyl-2-N,N,N-trimethylammonium chloride), and the mixture is suspended in aqueous medium to prepare cationic liposome. As the aqueous medium, a medium in which the obtained liposome can be stabilized, such as PBS, aqueous dextrose solution, or the like may be preferably used.

Step 2: Preparation of Expression Vector

Expression vector that expresses saxatilin is prepared by introducing cDNA of saxatilin gene to eukaryotic expression vector. An eukaryotic expression vector is preferably one selected from the group consisting of pAAV-CMV and pFLAG-CMV-1.

Step 3: Preparation of Lipoplexes

The cationic liposome and expression vector are mixed and homogenized in aqueous medium to prepare lipoplexes. As the aqueous medium, a medium in which the obtained liposome can be stabilized, such as PBS, aqueous dextrose solution, or the like may be preferably used. The mixed ratio of liposome to expression vector is from 2:1 to 20:1 (w/w).

Step 4: Injection of Lipoplexes

The lipoplexes are injected to cancer tissue.

Since the saxatilin gene treated according to the method described above effectively inhibits growth and metastasis of cancer tissue for a long period and does not give harmful effect on normal tissues, it can be widely used to effectively prevent and treat cancers.

Now, the present invention will be explained in terms of exemplary embodiments described in detail with reference to the accompanying Examples, which are given only by way of illustration, and it is obvious to a person having ordinary skill in the art that the scope of the present invention is not limited by these Examples of the present invention on account of the gist of the invention.

### EXAMPLE 1

### Expression and Concentration of Recombinant Protein

DOTAP and cholesterol were mixed in a ratio of 1:1 (v/v). The mixture was homogenized and suspended in physiological salt solution, to prepare cationic liposome having lamella structure.

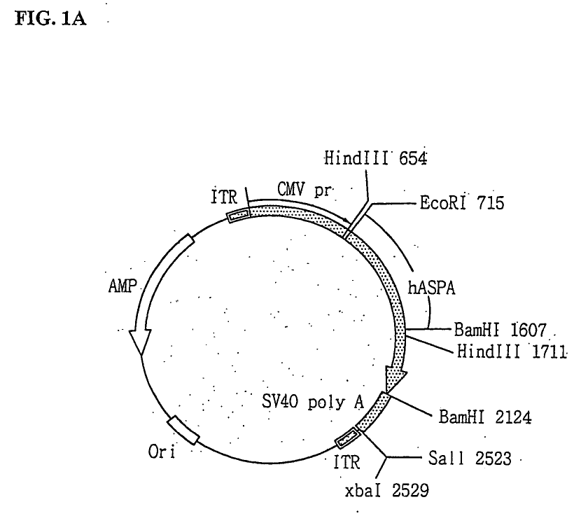

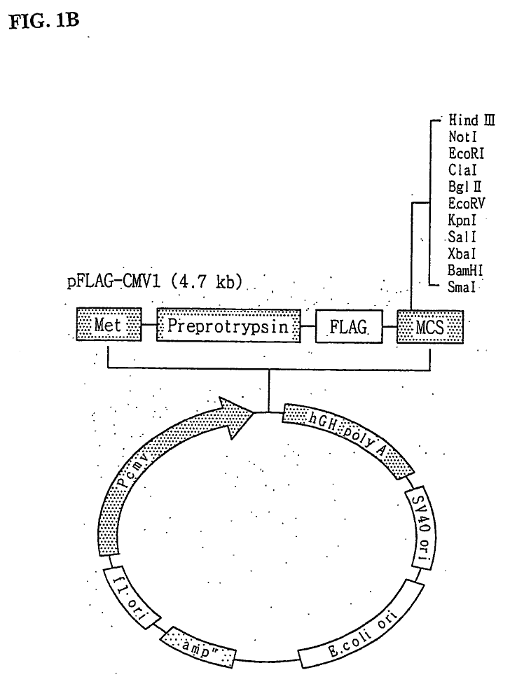

The cDNA of saxatilin gene was introduced to expression vector pAAV-CMV and pFLAG-CMV-1 (Sigma Chem. Co., U.S.A.) of FIG. 1 to prepare saxatilin expression vector pAAV-CMV-saxatilin and pFLAG-CMV-1 saxatilin, respectively. The cDNA of saxatilin gene was synthesized by polymerase chain reaction (PCR). As 5-terminal primer, CCCAAGCTTGCCACCATGGAGGCCGGAGAAGAATGT was used, while as 3-terminal primer, CGCGGATCCTTAGGCATGGAAGGGATT was used. The DNA fragment synthesized by polymerase chain reaction was digested by restriction enzyme, HindIII and BamHI and it was ligated into pAAV-CMV vector and pFLAG-CMV-1 vector which encodes FLAG (DYKDDDDK) epitope at the N-terminal (FIG. 2). Each expression vector thus prepared was introduced to E. coli DH5α to prepare transformant, and it was cultured. Each expression vector obtained therefrom was mixed with said cationic liposome in a ratio of 1:10 (w/w). The mixture was suspended in 5% (w/v) aqueous dextrose solution, and the suspension was homogenized to prepare lipoplexes. The lipoplexes were added to stabilization medium [DMEM (Dulbecos modified eagle's medium), 10 mM of sodium pyruvate, 500 U/ml of penicillin and 50 μg/ml of streptomycin], and the mixture was stabilized for 30 min at room temperature.

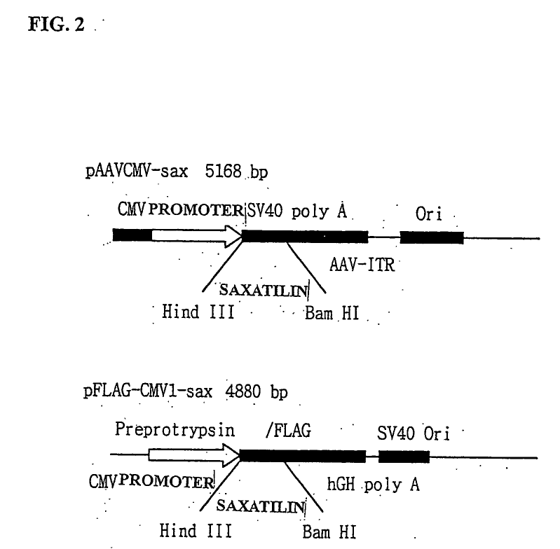

Eukaryotic 293 cells (American Type Culture Collection, ATCC) were incubated in a stabilization medium containing 10% (v/v) under CO₂ (5% v/v) condition, and said stabilized lipoplexes which comprises pFLAG-CMV-1 saxatilin as expression vector were injected. After replacing the medium with stabilization medium, the cells incubated under the same condition for 10 days. After cultivation, the 293 cell lysates were subjected to western blot by using anti-FLAG M2 antibody, so that it was confirmed that saxatilin was normally expressed, and that the expression vector normally expressed saxatilin in eukaryotic cells (FIG. 3).

### EXAMPLE 2

### Inhibition of Saxatilin Gene Against Cancer Cell Growth

To 50 μl of PBS containing 250 μg of cationic liposome, 25 μg of pAAV-CMV-saxatilin prepared according to Example 1 was added to prepare lipoplex, lipoACS.

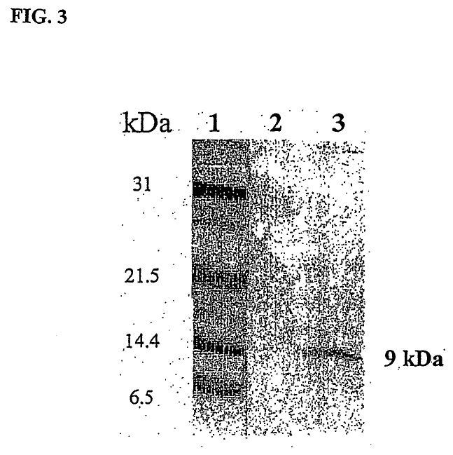

In the middle of the spine of C57BK16 mice bred for seven weeks, B16BL6 cells (5×10⁵), the lung cancer cells of mouse, were subcutaneously injected. The animals were bred to have the size of cancer tissue of 50 to 100 mm³, and divided into three groups. To the control group, PBS is intravenously injected by every 4 days, while to the experimental groups, lipoACS (25 μg/animal) was intravenously injected, and the size of tumor was measured as time passed (FIG. 4). As can be shown in FIG. 4, the increase rate of tumor volume considerably reduced in the experimental group of expressed saxatilin.

### EXAMPLE 3

### Effect of Saxatilin on Inhibiting Metastasis of Cancer

Lipoplex, lipoACS was prepared according to the same process of Example 2. A control group wherein PBS was subcutaneously injected to C57BK16 mice bred for seven weeks, and an experimental group wherein lipoACS (25 μg/animal) was injected, were prepared. To the tail vein of mice of each group, B16BL6 cells (4×10⁴) [lung cancer cells of mice] were injected every 5 days, and the mice were bred for 25 days until the mice of the control group died.

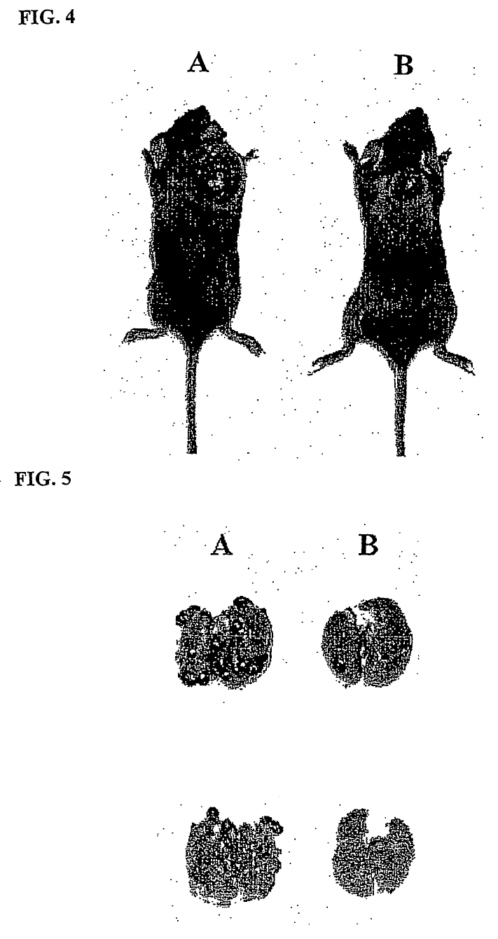

Then the number of colonies of lung tumor was calculated (see Table 1 and FIG. 5).

As can be seen from Table 1 and FIG. 5, the experimental group showed 85˜93% of metastasis inhibiting effect, as compared to the control group. Western blot using anti-saxatilin antibody was performed for the protein obtained from each experimental group. As a result, no saxatilin was detected in the control group while saxatilin was detected in the experimental group. Thus, the inhibition effect of the experimental group against cancer metastasis was the effect of saxatilin gene contained in the lipoplexes.

TABLE 1
Effect of saxatilin gene on inhibiting metastasis of cancer

| Plasmid containing | No. of | Average No. of colonies of | Inhibition |
|---|---|---|---|
| saxatilin gene | mice | lung cancer | (%) |
| pAAVCMV-Saxatilin | 3 | 7.3 ± 0.5 | 80.1 |
| Control | 3 | 30.3 ± 18.9 | 0 |

### EXAMPLE 4

### Effect of Saxatilin Expressed in 293 Cells on Inhibiting Bovine Capillary Endothelial (BCE) Cells Growth

BCE cells cultured in DMEM medium containing 10% of FBS was transferred to a flask to make the number of cells 4×10⁴/ml, and cultured in DMEM medium without FBS at 37° C. under 5% (v/v) CO₂ condition for 12 hours.

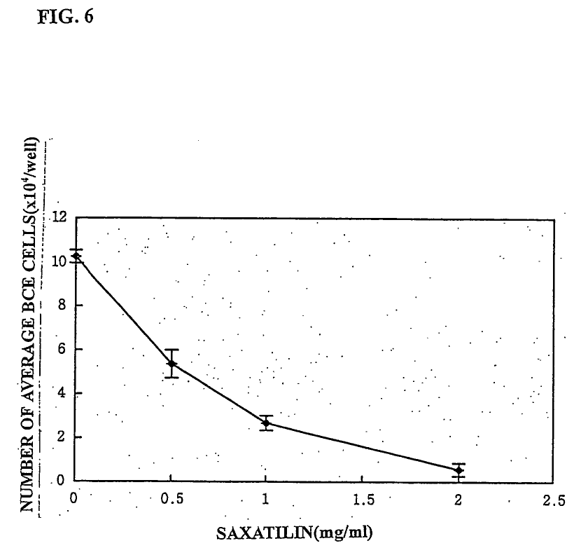

Then, it was treated with saxatilin expressed by pAAV-CMA-saxatilin expression vector in 293 cells. After 20 minutes, 1 ng/ml of bFGF was added and the mixture was cultured in DMEM medium containing 5% of FBS at 37° C. under 5% (v/v) CO₂ condition for 72 hours. Number of the survival cells was compared to measure growth inhibition rate. As can be seen from FIG. 6, growth of BCE cells was inhibited as being proportional to the amount of saxatilin added. At 2 μg/ml of saxatilin, 90% or more inhibition effect appeared.

As described above, the present invention provides a method for inhibiting growth of cancer cells by using saxatilin gene, which comprises steps of mixing cholesterol with DOTAP, suspending the mixture in aqueous medium to prepare cationic liposome and preparing expression vector for expressing saxatilin by introducing saxatilin gene to eukaryotic expression vector; mixing the cationic liposome and said expression vector in aqueous medium and homogenizing to prepare lipoplex; and introducing the lipoplex to a cancer tissue.

According to the present invention, a long-term and stable anticancer treatment can be performed by using saxatilin gene.

1. A lipoplex for inhibiting cancer growth, which comprises (a) cationic liposome and (b) expression vector containing saxatilin gene of sequence information 1.

2. The lipoplex according to claim 1, wherein said cationic liposome is one liposome selected from the group consisting of liposome containing DMDK and cholesterol, liposome containing DMEK and cholesterol, liposome containing DOTMA and cholesterol, liposome containing DC-Chol and DOPE and liposome containing DOTAP and cholesterol.

3. The lipoplex according to claim 1, wherein the expression vector is PAAV-CMA or pFLAG-CMV-1.

4. A method for inhibiting cancer growth by using saxatilin gene, which comprises steps of (a) mixing cholesterol with DOTAP, suspending the mixture in aqueous medium to prepare cationic liposome, and introducing saxatilin gene of sequence information 1 to eukaryotic expression vector; (b) mixing the cationic liposome and said expression vector in aqueous medium and homogenizing to prepare lipoplex; and (c) introducing the lipoplex to a cancer tissue.

5. The method for inhibiting cancer growth by using saxatilin gene according to claim 4, wherein the aqueous medium is phosphate buffer solution or aqueous dextrose solution.

6. The method for inhibiting cancer growth by using saxatilin gene according to claim 4, wherein the eukaryotic expression vector is PAAV-CMA or pFLAG-CMV-1.

7. The method for inhibiting cancer growth by using saxatilin gene according to claim 4, wherein the mixing ratio of liposome and expression is from 2:1 to 20:1 (w/w).


SEQUENCE LISTING (3 sequences)

SEQ ID NO: 1 - DNA, 222 bp, Agkistrodon saxatilis
gaggccggagaagaatgtgactgtggcgctcctgcaaatccgtgctgcgatgctgcaacctgtaaactgagaccaggggcgcagtgtgcagaaggactgtgttgtgaccagtgcagatttatgaaagaaggaacaatatgccggatggcaaggggtgatgacatggatgattactgcaatggcatatctgctggctgtcccagaaatcccttccatgcctaa

SEQ ID NO: 2 - DNA, 36 bp, Artificial Sequence
  5′ Primer for PCR of saxatilin gene
cccaagcttgccaccatggaggccggagaagaatgt

SEQ ID NO: 3 - DNA, 27 bp, Artificial Sequence
  3′ Primer for PCR of saxatilin gene
cgcggatccttaggcatggaagggatt